(19) United States
(12) Patent Application Publication
(10) Pub. No.: US 2021/0405992 A1
(43) Pub. Date: Dec. 30, 2021

(54) Managed Rooms Operational Maintenance

(71) Applicant: Microsoft Technology Licensing, LLC, Redmond, WA (US)

(72) Inventors: Sameer D. BEDEKAR, Issaquah, WA (US); Danilo PEREZ SOLANO, Bellevue, WA (US); David C. TSE, Woodinville, WA (US); Martin D. HALL, Sammamish, WA (US); David A. HOWELL, Seattle, WA (US); Irena K. ANDONOVA, Kirkland, WA (US); Jaya B. RAYASAM, Redmond, WA (US); Andrew A. GEORGE, Seattle, WA (US); Siddhartha ROY, Sammamish, WA (US)

(73) Assignee: Microsoft Technology Licensing, LLC, Redmond, WA (US)

(21) Appl. No.: 17/215,164

(22) Filed: Mar. 29, 2021

Related U.S. Application Data
(60) Provisional application No. 63/046,302, filed on Jun. 30, 2020.

(51) Int. Cl. G06F 8/65; G06F 8/71; G06F 9/48; G06N 20/00; G16H 40/40; G16H 40/63

(52) U.S. Cl. CPC G06F 8/65; G06F 8/71; G16H 40/63; G06N 20/00; G16H 40/40; G06F 9/4881

(57) ABSTRACT

A device-implemented method for providing managed services is described herein. An example method includes determining each managed device of a plurality of managed devices available in a shared space, and determining health data for the shared space. The health data describes properties of each managed device of the plurality of managed devices, including update information, performance information, configuration information, and security information. The method further includes identifying a problem with at least one of the plurality of managed devices, the problem being identifiable based on analysis of the health data for the shared space. The method also includes coordinating operational maintenance for the at least one of the plurality of managed devices to alleviate the problem. Coordinating operational maintenance may include performing at least two of a software update, a configuration change, and uninstalling software.

### BACKGROUND

Shared spaces are often equipped with dedicated audio, video, and computing equipment. For example, a shared space may be equipped with components such as audio, video, and computing equipment to enable collaboration between persons within the meeting room as well as persons located remotely from the meeting room. Equipping the shared space with the appropriate components can be challenging, as audio, video, and computing components are often not immediately compatible with other components to be used in the shared space. Moreover, each component may have differing update tasks, which can cause incompatibility across components of the shared spaces. This results in components of the shared space may becoming disabled or inoperable. In the event that a component of the shared space becomes disabled or inoperable, determining the root cause of these failures may prove difficult and time consuming.

### SUMMARY

The following presents a simplified summary of the subject innovation in order to provide a basic understanding of some aspects described herein. This summary is not an extensive overview of the claimed subject matter. It is intended to neither identify key or critical elements of the claimed subject matter nor delineate the scope of the subject innovation. Its sole purpose is to present some concepts of the claimed subject matter in a simplified form as a prelude to the more detailed description that is presented later.

An embodiment provides a device-implemented method for providing managed services. The method includes determining each managed device of a plurality of managed devices available in a shared space, and determining health data for the shared space. The health data describes properties of each managed device of the plurality of managed devices, including update information, performance information, configuration information, and security information, for example. The method also includes identifying a problem with at least one of the plurality of managed devices based on analysis of the health data for the shared space. The method also includes coordinating operational maintenance for the at least one of the plurality of managed devices to alleviate the problem. Coordinating operational maintenance may include performing at least two of a software update, a configuration change, and uninstalling software.

The following description and the annexed drawings set forth in detail certain illustrative aspects of the claimed subject matter. These aspects are indicative, however, of but a few of the various ways in which the principles of the innovation may be employed and the claimed subject matter is intended to include all such aspects and their equivalents. Other advantages and novel features of the claimed subject matter will become apparent from the following detailed description of the innovation when considered in conjunction with the drawings.

### BRIEF DESCRIPTION OF THE DRAWINGS

FIG. 1 is a block diagram of a networked environment including a managed rooms backbone;

FIG. 2 is a block diagram of a networked environment including a managed rooms backbone with managed rooms operational maintenance;

FIG. 3 is a process flow diagram of a method for a managed rooms backbone with operational maintenance;

FIG. 4 is a block diagram illustrating a system for processing and generating remediations;

FIG. 5 is a process flow diagram illustrating a method for processing and generating remediations;

FIG. 6 is a block diagram illustrating an exemplary computer readable medium encoded with instructions for a managed rooms backbone with operational maintenance according to aspects of the disclosed subject matter; and

FIG. 7 is a block diagram of an example of a computing system includes a managed rooms backbone with operational maintenance.

### DETAILED DESCRIPTION

As discussed above, a shared space may be equipped with audio, video, and computing equipment to enable collaboration between persons within the conference room as well as persons located remotely from the conference room. An organization often has multiple shared spaces, each with different equipment and capabilities. As used herein, a shared space is a collaborative meeting area with one or more components that enable information sharing. The shared space may be, for example, a boardroom, conference room, or huddle room. Each shared space may have a distinct environment, room size, layout, and purpose. Moreover, each shared space may have a distinct infrastructure. For example, a shared space may support wired or wireless networking, have a particular number of electrical outlets, have different acoustic characteristics, and the like. As used herein, the infrastructure of a shared space includes physical characteristics of the shared space that are difficult or impossible to change, as well as the network onto which the room hardware is connected. For ease of description, the present techniques may be described using a conference room as an exemplary shared space. However, the present techniques may apply to any shared space. In some embodiments, the shared space may be a shared virtual space, such as an online collaboration environment, online meeting platform, online social platform, and the like. Accordingly, the techniques described herein may apply to managed devices used in a personal work space to facilitate the virtual environment. The shared space may also be a commonly accessible communication network such as an Ethernet-based Local Area Network (LAN), a wireless LAN, a WiFi network, the Internet, Private Networks, Virtual Private Networks (VPNs), and others.

An organization is tasked with identifying the infrastructure of each shared space and equipping each space with audio, video, and computing equipment according to the identified infrastructure and desired capabilities of each space. Once shared spaces are equipped with these collaborative tools, the organization must monitor the equipment within in each space. Specifically, an organization must ensure that the equipment within each shared space is operable according to its intended function. Maintaining conference room equipment for multiple shared spaces is often difficult if not impossible due to each conference room having a different equipment and capabilities. Each conference room may include different cables, connectors, and other interconnects. Moreover, the different equipment can have dissimilar maintenance tasks, such as specific firmware updates, differing components that require monitoring, and the like.

For example, the managed devices within a shared space may each be subject to their own maintenance tasks to ensure proper operation of each device. Ensuring that each managed device is up to date and ready for operations involves several tasks performed at regular intervals. Moreover, an update for one device should not negatively impact the operation, performance, or security of another device. Thus, an organization should orchestrate management operations for each shared space to ensure the proper operation of each device in each shared space and prevent any updates from negatively impacting another device.

The present techniques enable managed rooms operational maintenance. As described herein, the managed rooms backbone performs operational maintenance on a target device to keep the target device operational, performant, and secure. In embodiments, a number of shared spaces are managed via a managed rooms backbone that comprises an agent that obtains and packages room health data signals, a transmitter to transmit the packaged room health signals, and cloud services to monitor shared space health and diagnose equipment problems or issues within the shared space. In embodiments, the agent may receive an update to be applied to a managed device such as a computing device, communication device, or peripheral component. In some cases, the agent may receive notice of an update to be applied to a particular managed device. The notice may include information describing the update that can be used to determine an impact of the update on other devices within a shared space. A relevance of each update to a particular problem may also be determined. The relevance of the update may include taking into account a risk assessment of performing one or more particular maintenance operations. Additionally, any missing updates are for managed devices may be determined. One or more updates may be applied via a staged roll-out. A staged roll-out is an update process wherein room system devices are grouped into update stages based on the risk-profile of that device. For example, some managed devices may be able to take high risk updates, wherein some managed devices can only take updates that have been thoroughly vetted. The staged roll-out system allows a customer to specify managed devices into update stages such that updates will be performed in different stages according to the determined risk profile.

In embodiments, conference room health is determined by analyzing key hardware signals, available operating system signals, and other application signals that are relevant to the conference room. In embodiments, the conference room health information may be combined and uploaded to a management cloud. The conference room health information may be collected, stored and analyzed/interpreted at the management cloud. The analyzed and interpreted conference room health information may be presented to a user, and the user may be directed to take actions within the conference room to remediate conditions that negatively affect the conference room health. The user may be a member of the organization that owns the conference room. In embodiments, remediation may be done remotely by a managed room service provider.

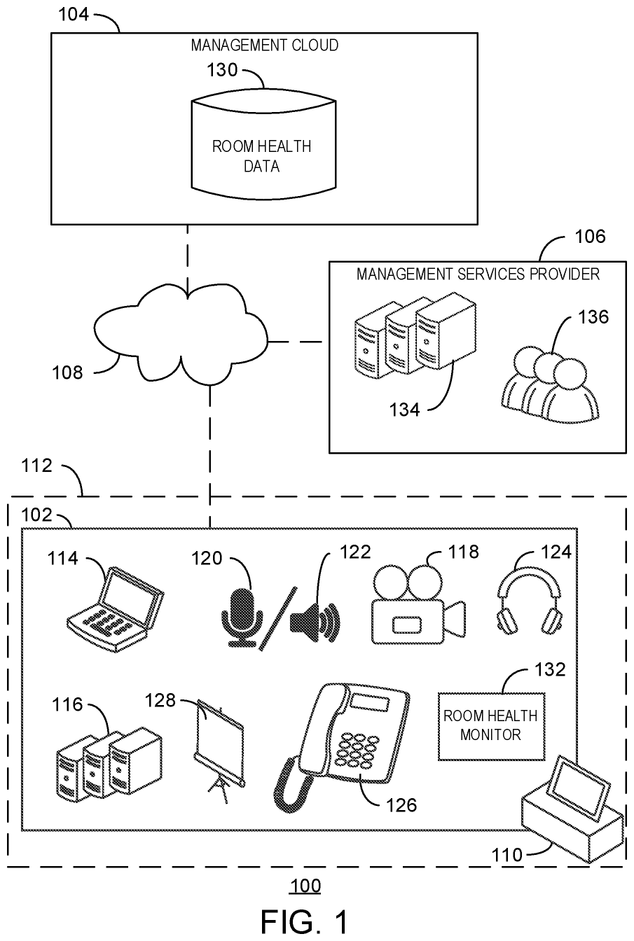

As a preliminary matter, some of the figures describe concepts in the context of one or more structural components, variously referred to as functionality, modules, features, elements, or the like. The various components shown in the figures can be implemented in any manner, such as via software, hardware (e.g., discrete logic components), firmware, or any combinations thereof. In some embodiments, the various components may reflect the use of corresponding components in an actual implementation. In other embodiments, any single component illustrated in the figures may be implemented by a number of actual components. The depiction of any two or more separate components in the figures may reflect different functions performed by a single actual component. FIG. 1, discussed below, provides details regarding one system that may be used to implement the functions shown in the figures.

Other figures describe the concepts in flowchart form. In this form, certain operations are described as constituting distinct blocks performed in a certain order. Such implementations are exemplary and non-limiting. Certain blocks described herein can be grouped together and performed in a single operation, certain blocks can be broken apart into plural component blocks, and certain blocks can be performed in an order that differs from that which is illustrated herein, including a parallel manner of performing the blocks. The blocks shown in the flowcharts can be implemented by software, hardware, firmware, manual processing, or the like. As used herein, hardware may include computer systems, discrete logic components, such as application specific integrated circuits (ASICs), or the like.

As to terminology, the phrase “configured to” encompasses any way that any kind of functionality can be constructed to perform an identified operation. The functionality can be configured to perform an operation using, for instance, software, hardware, firmware, or the like.

The term “logic” encompasses any functionality for performing a task. For instance, each operation illustrated in the flowcharts corresponds to logic for performing that operation. An operation can be performed using, for instance, software, hardware, firmware, or the like.

As used herein, the terms “component,” “system,” “client,” “server,” and the like are intended to refer to a computer-related entity, either hardware, software (e.g., in execution), or firmware, or any combination thereof. For example, a component can be a process running on a processor, an object, an executable, a program, a function, a library, a subroutine, a computer, or a combination of software and hardware.

By way of illustration, both an application running on a server and the server can be a component. One or more components can reside within a process, and a component can be localized on one computer and/or distributed between two or more computers. The term “processor” is generally understood to refer to a hardware component, such as a processing unit of a computer system.

Furthermore, the claimed subject matter may be implemented as a method, apparatus, or article of manufacture using standard programming and/or engineering techniques to produce software, firmware, hardware, or any combination thereof to control a computer to implement the disclosed subject matter. The term “article of manufacture” as used herein is intended to encompass a computer program accessible from any computer-readable storage device or media.

Computer-readable storage media can include but are not limited to magnetic storage devices (e.g., hard disk, floppy disk, and magnetic strips, among others), optical disks (e.g., compact disk (CD) and digital versatile disk (DVD), among others), smart cards, and flash memory devices (e.g., card, stick, and key drive, among others). In contrast, computer-readable media (i.e., not storage media) generally may additionally include communication media such as transmission media for wireless signals and the like.

FIG. 1 is a block diagram of a networked environment 100 including a managed rooms backbone with managed rooms operational maintenance. In the environment 100 a managed rooms backbone 102, management cloud 104, and management services provider 106 are illustrated. Each of the managed rooms backbone 102, management cloud 104, and management services provider 106 may be communicatively coupled via a communication network 108. The managed rooms backbone 102 may execute via a room management device 110. In examples, the room management device 110 may be a hub that enables video conferencing and collaboration. In particular, the room management device 110 can enable room scheduling, screen sharing, and video conferencing. In embodiments, the room management device 110 is communicatively coupled with a number of managed devices via the managed rooms backbone 102.

As described herein, the managed rooms backbone 102 is a framework that interconnects managed devices and networks to achieve seamless collaboration via the managed device functionalities across one or more networks. The managed rooms backbone 102 monitors communication between the managed devices and networks. The managed rooms backbone 102 may also monitor communication with equipment/managed devices in other conference rooms. In embodiments, the managed rooms backbone 102 may function as an interface between a user and each managed device.

For example, the managed rooms backbone 102 may execute on the room management device 110 and monitor communications between various devices within a conference room 112. The room management device 110 may be a special purpose device with custom hardware and/or software components, as well as a desktop computer, a laptop computer, a tablet computer, a smart phone, and a wearable computing device with custom components among other similar devices. The room management device 110 may, for example, managed devices 114, 116, 118, 120, 122, 124, 126, and 128. The managed devices 114-128 may be collocated within a same conference room or shared space. The managed rooms backbone 102 along with the managed devices 114-128 enable users to share content, interact and communicate with one another, create and share calendars, and schedule meetings, among other things, through various communication modes, such as e-mail, text message, telephone, video conferencing, and the like. The room management device 110 may monitor and manage meeting functionality as enabled by each of the managed devices 114-128. For example, the room management device 110 may provide a user interface to access or initiate a meeting and manage meeting features such as audio/video controls, presentations, recordings, and the like as enabled by the devices 114-128. The room management device 110 may communicate with the managed devices 114-128 via short range wireless communication such as near field communication (NFC), Bluetooth communication, personal area network (PAN) communication, optical communication, and the like. The room management device 110 may also communicate with the managed devices 114-128 via wired communications, for example, universal serial bus (USB) or similar connections.

As illustrated in FIG. 1, the managed devices 114-128 include computing devices such as a laptop 114 and one or more servers 116. In examples, the laptop 114 and servers 116 may enable computing functionality within a conference room or shared space that is monitored by the room management device 110 via the managed rooms backbone 102. In embodiments, the laptop 114 and servers 116 may be shared computing devices that are associated with the particular conference room or shared space. In operation, any user within the conference room or meeting space can access the laptop 114 or servers 116 for any number of computing functions while in the conference room or shared space.

The cameras 118 enable the capture of video or images within the conference room or shared space. In embodiments, the cameras 118 may be fixtures within the conference room or shared space. In particular, the cameras 118 may be strategically placed such that all meeting spaces within the conference room or shared space can be captured. In embodiments, the cameras 118 may include pan, tilt, and zoom functionality to enhance image and video capture within the conference room or shared space. For example, each camera 118 may have a range of movement such that the conference room can be captured in its entirety. Each camera 118 may also be able to zoom in on an area, including zooming in on an individual speaker. The particular lenses and other components of the cameras 118 may be selected according to their suitability for use within a particular conference room for meeting space. For example, to capture a conference room in its entirety, a camera 118 may include a wide-angle lens.

Managed devices within a conference room or shared space can also include microphones 120 and speakers 122. In embodiments, the microphones 120 and speakers 122 may be physically integrated with each camera 118. Additionally, the microphones 120 and speakers 122 may be individual components strategically placed within the conference room or shared space. In embodiments, each microphone 120 may be positioned within the conference room or shared space such that sound is captured from all locations within the shared space. Further, each speaker 122 may be positioned within the conference room or shared space such that sound is adequately provided to all locations within the conference room or shared space.

Managed devices can also include headsets 124. The headsets 124 may be used to enable private calling or conferencing within a conference room or shared space. In embodiments, speakerphones 126 may be used to access telephone functionality within the conference room or shared space. In embodiments, the speakerphone 126 functionality may be integrated with the conference room device. In this manner, other meeting rooms and persons may be accessed from the conference room or shared space. One or more screens 128 can be used to share content or view other meeting participants. In embodiments, the screens may be a screen, display device, television monitor, computer monitor, and the like.

While particular managed devices have been illustrated as collocated in an exemplary conference room 112, any number or type of managed devices can be included in the conference room. Moreover, in addition to the managed devices, the conference room or shared space may also include other devices and connectors such as mounting devices, cables, hubs, and other devices used to connect the managed devices to enable a seamless audio/video experience. In embodiments, the other devices may be selected to enable interoperability between managed devices. The devices of a conference room may also be used to measure the acoustics of the room and the quality of the network connection to the conference service. The room acoustics and network connection can affect the meeting experience within the conference room and can be measured and analyzed as a component of the conference room health. Some properties of the shared space may also be entered by user input, such the size of a room, the arrangement of seats within a room, and others.

In embodiments, the managed rooms backbone 102 may monitor hardware signals from each managed device 114-128 to generate health data. In addition to hardware signals associated with each managed device, the managed rooms backbone 102 may also monitor operating system signals associated with the managed devices to generate health data. Moreover, the managed rooms backbone 102 may capture signals from other applications executing on devices within the conference room or shared space. For example, data from other applications may include managed device supplemental data and a telemetry heartbeat. The managed rooms backbone 102 may also query hardware signals that are specific to a particular device type. For example, query application programming interfaces (APIs) from a particular hardware manufacturer to obtain a more specific error code or signal associated with the particular device. The managed from backbone 102 may combine this information and securely transmit the information across the communication network 108 to the management cloud 104. The combined data signals are generally referred to as room health data. In embodiments, this information may be selectively combined or packaged to reduce the transmission of redundant signals or signals that are presently irrelevant to room health.

The health data may be characterized as a set of properties that apply to the shared space, including properties of specific managed devices or combinations of managed devices within the shared space. The combination of properties describes the health status of the monitored system. The health data can include properties of the shared space such as room size, networking capabilities within the room such as bandwidth, projection screen size, acoustic qualities of a room, synchronization of a video device with an audio device, potential audio feedback from multiple nearby devices, number of seats, distance of seats from screen, and others. The health data can also include an identification of all of the electronic devices within a shared space and various properties of each of the devices. Examples of such properties include hardware and software configurations, performance properties, error codes generated by software running on managed devices, location of the managed device, security information, and others. Examples of hardware or software configurations properties can include update status information, software or firmware versions operating on managed devices, driver versions, operating system versions, and others. Performance properties may include performance information such as processor use percentage, working memory use percentage, storage disk reads and writes, network throughput, and others. Examples of security properties include information such as measurement of threats such as malware and viruses, existence of vulnerabilities such as known Common Vulnerabilities and Exposures (CVE) bulletins, and missing patches for first-party and third-party software, and first-party and third-party driver and firmware. Continuous monitoring of the health data enables data to be collected regarding the history of the device, past performance of the device, and the like.

In embodiments, the room health monitor 132 may execute via the room management device 110. In embodiments, the room health monitor 132 may analyze the obtained room health data to determine the health of the conference room 112. As used herein, health generally refers to the fitness of the conference room for a shared, collaborative user experience. The general health of the conference room is based on, at least in part, the health of the room management device 110, managed devices 114-128, and other devices including each connection that enables interoperability across the room management device 110 and managed devices 114-128. The room health monitor may assess a number of health indicators based on the received room health data. This assessment may be an interpretation of the health of the conference room and any devices within the conference room.

The management cloud 104 includes a room health data store 130 to store room health data. In embodiments, the assessment of room health is provided to management service provider operators or to information technology (IT) administrators within the customer's organization. As used herein, the on-site user may be located within the conference room 112 and can view the assessment of room health via the room management device 110. In embodiments, the assessment of room health for a conference room can be viewed by a user that is a member of the organization at any location within the organization. Thus, the analyzed room health data may be provided via a dashboard of the managed rooms backbone 102 that executes on the room management device 110. The analyzed room health data may also be provided via the dashboard that executes on another device within the organization. In the event that the room health status indicates an issue or failure of components within the conference room, the management cloud 104 may also perform operational maintenance by transmitting remediation tasks or strategies to a user. In some cases, a management services provider 106 may perform remote remediations of the conference room 112. Remote remediations may be executed automatically via remediation servers 134. Remediation experts 136 may also access the room health assessment and provide remote remediations. In various scenarios, the failure may be due to a single electronic device or due to interactions between two or more electronic devices. For example, a configuration or update applied to one electronic device may cause a problem to arise between it and other electronic devices in the shared space, such as an inability to communicate or properly process data transferred between devices. Remediations may be based on a holistic analysis of the health data that is able to identify problems related to more than one component of the shared space. The holistic analysis of the health data enables the identification of problems that may be due to interactions between electronic devices rather than a single point of failure.

In embodiments, remediations may include restoring the original configuration of the managed devices in the conference room. This may include, for example, ensuring cable connections are intact. Remediations may also include managed device-specific tasks, such as updating drivers or firmware. Other exemplary remediations include operating system remediations, such as setting power plans, lockdown policies, updating and applying operating system hotfixes and security policies, and the like. Application remediations may include comparing application configuration to a physical configuration, for example, comparing display configuration as expected by an application to the actual physical configuration visible to those in the conference room. In this example, the application may intend to project to two front of room displays but in reality, there is only one display. Remediations in this example include detecting the discrepancy and correcting the application configuration as needed.

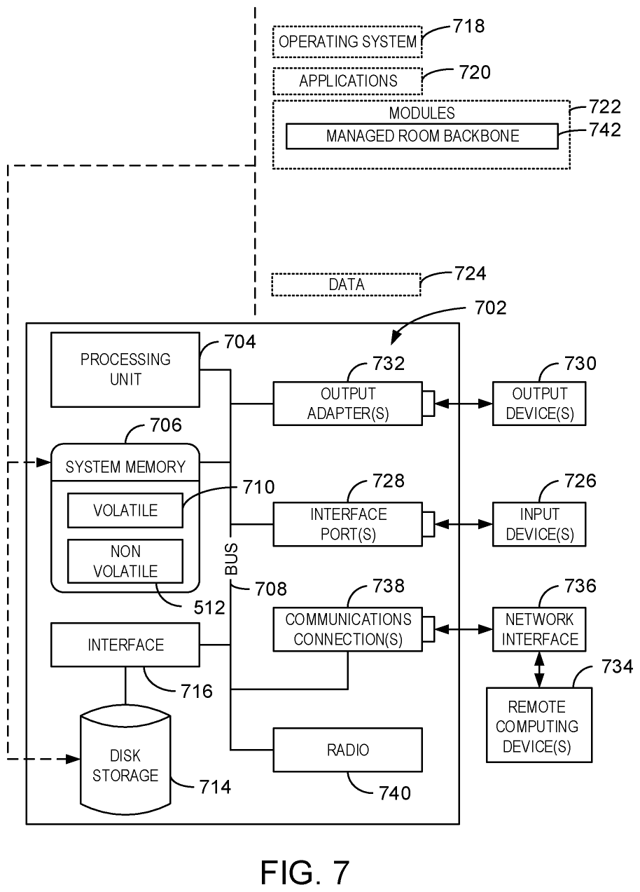

The block diagram of FIG. 1 is exemplary and should not be viewed as limiting to the managed rooms backbone or networked environment. Note that the managed rooms backbone or networked environment can have more or fewer blocks than those illustrated in the example of FIG. 1. Moreover, some blocks may be implemented via a computing device such as the computing device 700 of FIG. 7. Additionally, it will be appreciated that the techniques described herein may also apply to operational maintenance of a shared virtual space, such as an online meeting environment. Accordingly, the techniques described herein may apply to managed devices used in a personal workspace to facilitate a virtual collaboration environment.

As generally discussed above, remediations may also include managed device-specific tasks, such as updating drivers or firmware. Keeping a target device up to date and ready for operations involves several tasks that need to be performed regularly. The managed rooms backbone may be used to orchestrate operational maintenance on each device to ensure the target device is operational, performant, and secure. In particular, the present techniques go beyond simple automatic execution of updates. Instead, the present techniques coordinate a particular schedule for each update in view of other updates and devices. In this manner, the present techniques can combine updates amongst one or more devices into blended tasks that have complex dependencies. Dependencies could include multi-step task sequences that must be executed sequentially or have pre-requisite conditions such as existence of a particular driver or managed device, for example. Dependencies can include conditions existing at runtime, such as registry corruption. Blended tasks will incorporate these complex dependencies into their eligibility targeting. Combining tasks into blended tasks simplifies the update process by recognizing the common processes and tasks among the updates. The updates may also be verified such that they do not negatively impact another device managed by the managed rooms backbone.

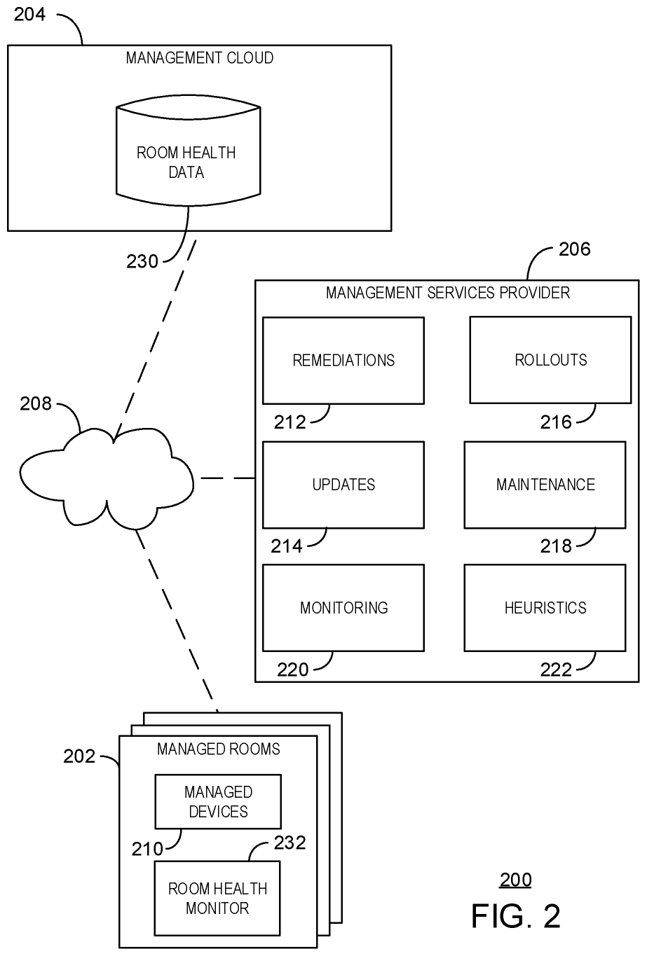

FIG. 2 is a block diagram of a networked environment 200 including a managed rooms backbone with managed rooms operational maintenance. In the environment 200 a managed rooms backbone 202, management cloud 204, and management services provider 206 are illustrated. Each of the managed rooms backbone 202, management cloud 204, and management services provider 206 may be communicatively coupled via a communication network 208. The managed rooms backbone 202 may manage one or more managed devices 210 physically located in a respective managed room 202. In examples, each managed room 202 includes a room management device that is configured as a hub that enables video conferencing and collaboration. In particular, the room management device can enable room scheduling, screen sharing, and video conferencing. In embodiments, the room management device is communicatively coupled with a number of managed devices 210 via the managed rooms backbone. The managed room also includes an instantiation of a room health monitor 232. In embodiments, the room health monitor 232 may analyze the obtained room health data to determine the health of the conference room 212. Additionally, the management cloud 204 includes a room health data store 230 that stores the health data such as the health data described above.

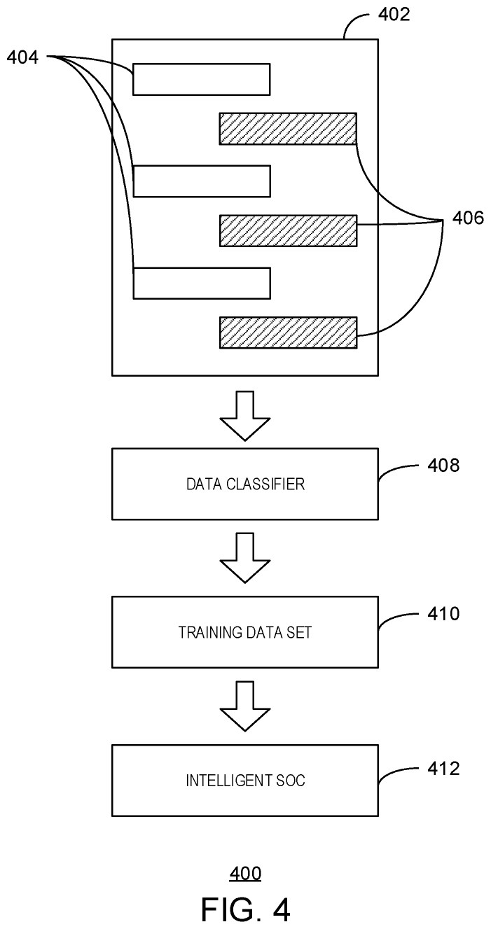

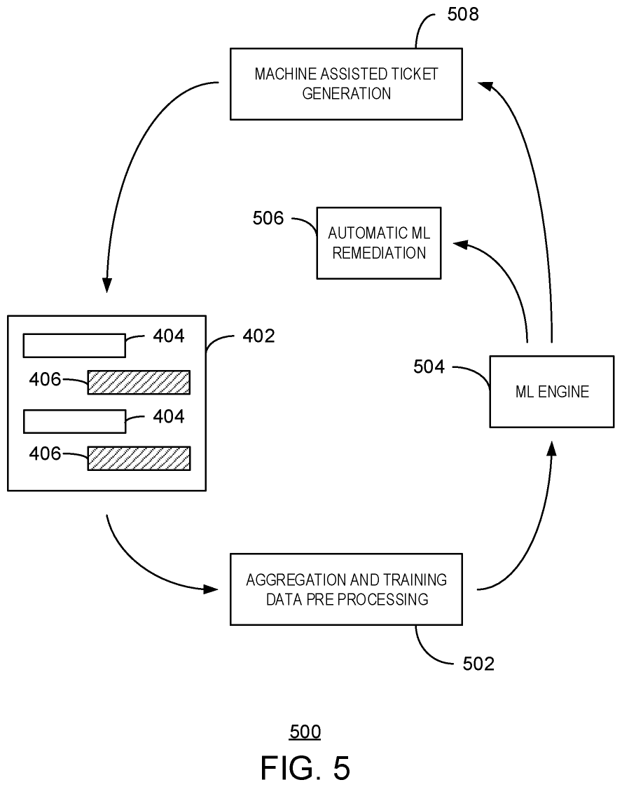

In embodiments, a management services provider 206 may configure and perform remediations 212 of the conference room based on analysis of the health data, which may be performed by human agents or automatically via a machine learning system, for example. In some embodiments, one or more human agents can act as remediation experts that analyze room health data and generate remediations based on expert knowledge about the managed devices and the nature of the problems being encountered. Additionally, remediations may also be configured automatically by a machine learning system trained to recognize patterns in the health data and associate specific patterns with specific remediations. Techniques for automatically generating remediations are described further in relation to FIGS. 4 and 5.

A remediation may be configured as a set of tasks to be performed by a user or automatically. Remediations may be executed automatically via remediation servers. For example, the performance of a remediation may include sending a script (e.g., PowerShell script) to the room management device 110 (FIG. 1) to run locally and execute a set of tasks associated with the remediation. Remediations may also include managed device-specific tasks, such as updating drivers or firmware. In embodiments, updates 214 may be executed to ensure proper operation of each device. As used herein, an update may refer to improved or fixed software that is to replace older versions of the same software. In this manner, a software update may bring devices current with the latest security software, drivers, and system utilities. To determine whether a particular update is likely to remedy a particular problem, an update relevant may be determined for each available update. The update relevance may be determined based on a number of factors, including which OEM systems the update qualifies for, which source drivers are present, what combinations of software are present that necessitate a particular patch, what runtime characteristics associated with the problem are occurring, and others.

In embodiments, an update may also refer to the replacement of a physical device managed by an instance of the managed rooms backbone 202. In some embodiments, the remediation may be to roll back software or firmware to a previous version. Rolling back to previous version may alleviate problems caused when the newer version is discovered to have a coding error, or when the newer version is discovered to be incompatible with other devices or software/firmware executing on other devices. Remediation tasks may also include changing a configuration setting of a managed device to keep the room healthy and functional, such as setting USB power states for more reliable operation, setting display settings (refresh rate, DPI, and resolution), resetting volume, and the others.

In some embodiments, the remediation may include a set of common tasks, which are tasks to be performed for each one of a plurality of managed devices. Examples of common tasks include software updates that are to be performed on several computing devices to ensure that each computing device is running the same version of the software. A remediation can also include combinations of different tasks that may be performed, such as performing a software update in addition to setting configuration parameters, and the like. The combination of tasks to be performed may be referred to herein as a blended task.

In embodiments, the management services provider 206 may also enable monitoring 220 of the managed rooms via the managed room backbone 202. Monitoring may include observation of one or more performance parameters in order to maintain and enhance the performance of each device managed via the managed room backbone 202. Additionally, maintenance 218 may be enabled in order to maintain all the configurations required for each device in a managed room in order to maintain proper functionality of the device and correct any configuration drift as described below. When updates are applied to devices, a rollout mechanism 216 may be enabled to configure staged rollouts of one or more updates.

In embodiments, heuristics 222 may use a combination of software, human intelligence, and machine learning to capture base knowledge information to be used in the management of devices in a shared space. In determining the heuristics described in several of the monitors and detectors, the managed rooms backbone may use a combination of software, human intelligence in a Service Operations Center (SOC) and machine learning. For example, the SOC will monitor and look for patterns taking into account inventory, what actions were performed, as well as knowledge base information. The SOC may also keep regular sync meetings with Operation System partners (such as Windows and Android), OEMs (such as hardware audio/video peripheral and computer OEMs). This will facilitate knowledge of their problem/investigation pipeline, knowing what fixes they will be making available, and helping to determine risk and targeting. This knowledge may be codified into a knowledge base that includes Standard Operating Procedures, Troubleshooting Guides, Support Capabilities, and a backlog representing items needed to be added to the software heuristics and remediations.

In embodiments, the managed rooms backbone will monitor current inventory as well as individual signals on the target device. The software will also monitor virus/malware patterns. The managed rooms backbone machine learning algorithms may combine inventory, applied maintenance updates and operations, and signals that are produced as a result to determine success and risk factors of individual updates and maintenance operations.

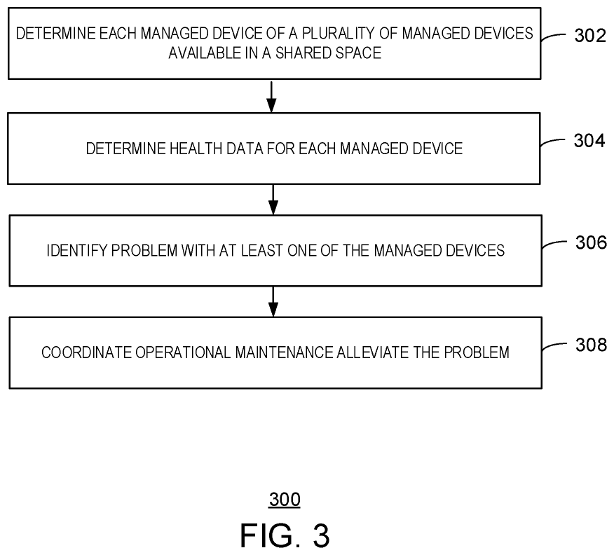

FIG. 3 is a process flow diagram of a method 300 for a managed rooms backbone with managed rooms operational maintenance. In embodiments, the method 300 enables the orchestration of management operations on behalf of an organization.

At block 302, each managed device of a plurality of managed devices available in a communication network is determined. The managed devices may include any type of managed device used to facilitate communication and collaboration in a shared space, including a shared virtual space. The managed devices may be any of the devices described in relation to FIG. 1 and others.

At block 304, health data for each managed device is determined. The health data can include information such as update information, performance information, configuration information, and security information. The health data may be collected by analyzing hardware signals from each of the plurality of managed devices as described above. The health data describes properties of each managed device in the shared space, and may include data comprising update information, performance information, configuration information, and/or security information.

At block 306, a problem with at least one of the managed devices is identified. The problem may be identifiable in at least one of the update information, the performance information, the configuration information, or the security information. For example, the managed rooms backbone may determine that an update is available for a specific managed device and that the current version operating on the managed device is out of date.

In some embodiments, the managed rooms backbone will use heuristics to determine when a software update is missing based on factors such as versions of existing drivers and other software on the device. The managed rooms backbone can look across “store” apps, apps that manifest in “add/remove programs”, apps that show in operating system features such as “windows features”, and apps that appear as drivers in the appropriate “Device Manager” for that operating system. Software updates of components within a target device can include security fixes, maintenance updates, bug fixes, performance fixes, and more. Updates can come from the Operating System, OEM manufacturers, connected peripheral (such as Audio/Video, USB extenders, and more) manufacturers, network drivers, and other factors. The updates may be received periodically, at known intervals. The updates may also be received in response to a known issue or defect with previous software versions or the physical device.

Upon receiving updates, the relevance of each update may be determined. Determining the relevance may include ascertaining hardware that is on the device as well as combinations of hardware. For example, certain functions of the device such as HDMI Ingest require specific versions of software across both the BIOS as well as a chipset driver. The update will have targeting metadata to ensure it is applied only to eligible machines.

Other type of problems detected at block 306 may include communication problems between two different managed devices, security weaknesses, slow performance, inoperability of one or more managed devices, incompatible software versions on different managed devices, incompatible communication protocols between different managed devices, and others. In some embodiments, the problem may be identified automatically, by inputting the health status to a machine learning engine, as described further in relation to FIGS. 4 and 5.

At block 308, operational maintenance is coordinated for at least one of the managed devices to alleviate the problem. Coordinating operational maintenance may include performing at least two of a software update, a configuration change, and uninstalling software. Operational maintenance may be performed by providing notice of the problem to an agent or implementing a remediation, which includes one or more tasks to be performed for the components of the shared space. In some examples, remediation tasks to be performed are identified automatically, and can be output by a machine learning engine as described further in relation to FIGS. 4 and 5. A remediation can include combinations of various types of remediation tasks. One type of remediation task is a software update task. In such cases, analysis may be performed to ensure that updates propagated across multiple managed devices and multiple types of managed devices will be compatible.

Coordinating operation maintenance can include determining possible risks involved in implementing the update. As used herein, risk is a probability of a negative impact on the managed rooms system. In embodiments, the risk of the update is ascertained in order to determine an update strategy. In embodiments, a managed rooms backbone team may determine risk of the update. An update may be determined low risk based on how dependent it is on diverse environmental factors. For example, if an update requires internet to apply, it is deemed higher risk than an update which is completely self-packaged. If an update has dependencies on particular OS feature updates to be installed, it is higher risk than an update which does not have such dependencies. Several factors including dependency trees and change of state after the update determine its risk. In embodiments, the operations team will test updates in a lab to ensure compatibility across risk factors.

Coordinating operation maintenance can also include the applying the update via a staged rollout. In embodiments, the staged roll-out may be according to the determined update strategy. This strategy will include identification of devices by the customer as well as the assessed risk by the Managed Room Operators. An organization may stage roll-outs in order to monitor the progress of the roll-outs carefully. The managed rooms backbone enables organizations to set a roll-out policy by specifying that machines to be assigned into “rings” of deployment. Rings are used to deploy software progressively. In embodiments, the managed rooms backbone will default to three rings 1) Staging; 2) General; and 3) Executive. The staging ring represents early machines for rollout and validation. The general ring is then the general population of target devices. The executive ring represents executive rooms that are the last rooms to be rolled out. In embodiments, the managed rooms backbone supports two settings per deployment ring 1) Roll-out duration; and 2) a test period. The roll-out duration specifies across what time period a roll-out should occur. For example, if a roll-out period is two weeks, and there are fifty-six machines in that deployment ring, then the managed rooms backbone system will deploy updates on average to four machines per day.

In embodiments, the managed rooms backbone may employ various roll-out strategies based on the risk of a particular update. In a linear strategy, the roll-out will happen equally distributed throughout the roll-out period. For example, a linear strategy would be four machines per day if there are fifty-six machines in two weeks. In a higher risk roll-out, it may happen exponentially in order to reduce risk. For example, exponentially there may be one machine per day for four days, then two for two days, then four machines per day, and so forth until complete. The managed rooms backbone will determine the appropriate time window to apply the updates, including factors such as a defined maintenance window, defined business hours, and required number of reboots to determine when best to apply the updates.

Remediation tasks can also include various types of configuration tasks. In embodiments, the managed rooms backbone may monitor and maintain all key configurations required on the device in order to keep it functioning and correct any “configuration drift.” As used herein, configuration drift is when software intentionally or inadvertently changes settings that may be required. For example, a policy push may apply power saving settings across an organization. However, conferencing devices have unique power plans specific to conference rooms (such as specific always on settings) that will be incompatible with the power saving policy. This is undesired configuration drift.

The managed rooms backbone may also monitor and maintain/remediate configuration intended for lockdown of a device, operations, and security. For example, lockdown of device refers to the appropriate settings for lockdown of the device that allows the device to act as a purpose-built kiosk. For example, hiding of the start menu, hiding of the search menu, hiding of the desktop, disabling of certain keyboard combinations such as “alt-tab” and “alt-F4” which would allow a user to break out of the conferencing experience.

Furthermore, the managed rooms backbone may maintain/remediate configurations that impact operations of the conferencing software. This includes settings that allow the conferencing software to perform appropriately (such as on-screen keyboard settings, resolutions, selected/default audio/video, peripherals, power plan settings, and more). The managed rooms backbone will also maintain/remediate external configurations that impact operations of the conferencing software, such as an alert on an external configuration. The managed rooms backbone will also maintain/remediate configurations intended for security monitoring and analytics. In particular security monitoring and analytics includes anti-virus settings such as what software is monitored, where signature and application updates come from, malware detection settings, and other relevant settings.

The managed rooms backbone may also determine and remove software that is not relevant or that may be detrimental to the device. The managed rooms backbone will perform preventative health measures, such as disabling automatic updating of specific software that is not relevant or compatible with the device. Additionally, the managed rooms backbone will employ intelligent software to determine heuristics above and use machine learning to find patterns of configurations that have caused problems in the past in order to flag and examine as well as remediate configuration drift. In embodiments, the managed rooms backbone may perform monitoring and maintenance in order to maintain and enhance performance of the device. This includes but is not limited to installed applications, executing applications, registry, hard disk performance, and central processing unit utilization. For example, installed applications may be monitored to ensure the correct application set is installed, and that extraneous applications are not installed. Mitigating actions may also be performed, such as uninstalling software. Executing applications may be monitored to ensure the correct applications are running, and that extraneous applications are not running Mitigating actions, such as stop/kill of apps that should not be running, may be performed. Monitoring may also include monitoring the size of the registry and determining where it may be changing through snapshots and analysis of the snapshots. Additionally, performance of the hard disk drive, including fragmentation status, free space, and metrics such as Reads/Writes per second, cache utilization, and more may be monitored. Mitigating actions such as deleting caches, temporary files, unneeded software, and invoking defragmentation operations on the drives may be performed. Overall CPU utilization over time and CPU utilization of executing application may also be monitored. In some examples, the application inventory may be compared against baseline of expected software required to keep the device operational. Drivers, proxy settings, and e-CDN settings specific to intensive operations such as video rendering, and audio/video streaming may also be monitored.

The managed rooms backbone may be implemented as a service. For example, the managed rooms backbone may be a single point to determine failures and coordinate responses (across OEMs, support, customers, third party software, and more).

FIG. 4 is a block diagram illustrating a system for processing and generating remediations. The system 400 may be implemented by a management services provider such as the management services provider 106 shown in FIG. 1 or the management services provider 206 shown in FIG. 2. The management services provider is configured to receive health data from one or more managed systems such as the managed rooms 112 of FIG. 1 or the managed rooms 202 of FIG. 2.

Based on analysis of the health data, operations tickets 402 can be generated. Operations tickets may be generated and populated by human operators when attempting to solve a problem with a managed system or provide some type of operational maintenance of the managed system. The operations ticket 402 describes the properties of a remediation performed on the managed system such as elements of the health data that caused the operations ticket to be generated and tasks that were performed to mitigate any problems. The operations ticket 402 can include informational inputs 404 entered by the operator such as a problem description, actions taken to mitigate the problem, how the problem was resolved, and the like. The operator also associates each informational input 404 with a classification tag 406 that enables data to be classified by the human agent as it is entered. Each classification tag may be a code that applies to the specific type of data being entered by the human agent.

Operations tickets 402 can be input to a data classifier 408 that sorts and structures the data to generate a training data set 410. Once a sufficient set of operations tickets 402 have been generated, the data classifier 408 will be able to predict appropriate remediations for particular sets of input data.

The training data set 410 can then be used to train an intelligent service operations center (SOC) 412 using machine learning algorithms. The intelligent SOC 412 has the ability to process health data to identify patterns in the data and automatically apply remediations. As more human generated operations tickets are entered into the system, the intelligent SOC's ability to automatically recognize and fix problems increases to the degree that many remediations may be handled fully automatically without the involvement of human operators.

FIG. 5 is a process flow diagram illustrating a method for processing and generating remediations. The method 500 may be performed by the system 400 described above. The method 400 can be performed on an ongoing basis to generate continuously updating set of information and actions as health data is received and operations tickets 402 are generated.

At block 502, an operations ticket 402 is received and aggregation and training data preprocessing is performed. As described above, the operations ticket 402 include informational inputs 404 each of which is associated with a specific classification tag 406. The aggregation and preprocessing adds the data associated with the operations ticket to the body of training data that has been collected.

At block 504, the training data is used to train a machine learning engine. The machine leaning engine may be retrained periodically as the new operations tickets become available and the body of training data expands. After each training cycle, the machine learning engine will be able recognize a larger body of problems and respond automatically with a larger suite of automatic remediations.

At block 506, the machine learning engine can generate an automatic remediation. To generate a remediation, a particular subset of the monitored health data applicable to a specific monitored system may be input to the machine learning engine. The output of the machine learning engine may be a particular set of tasks to be performed in response to the health data as part of the remediation.

At block 508, a machine assisted operations ticket is generated. The machine assisted operations ticket is an operations ticket that is partly filled out by the machine learning engine. For example, in some cases, the machine learning algorithm may not have a solution to a problem indicated by the input health data. This may happen if a new problem arises that has not yet been addressed by a human operator and no relevant examples have been entered into the body of training data. In such cases, a human operator may need to review the health data to arrive at a suitable remediation. However, the machine learning engine may be able to recognize patterns in the health data that are relevant to the solution. Accordingly, the machine learning engine may generate an operations ticket that is at least partly filled out with information such as a description of the problem and any relevant health data indicative of the problem. The machine assisted ticket can then be sent to a human operator for further analysis. Once the human operator has formulated a remediation and completed the ticket, the process flow can return to block 502 and the operations ticket can be added to the body of training data.

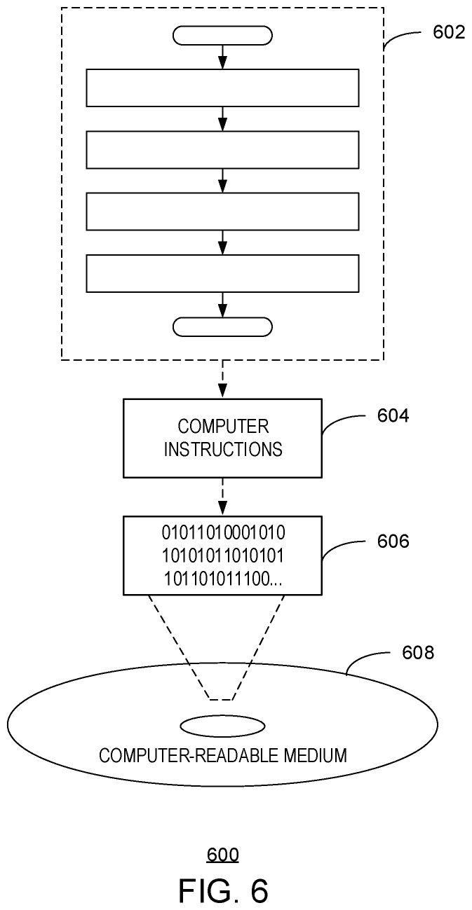

Turning to FIG. 6, FIG. 6 is a block diagram illustrating an exemplary computer readable medium encoded with instructions for a managed rooms backbone according to aspects of the disclosed subject matter. More particularly, the implementation 600 comprises a computer-readable medium 608 (e.g., a CD-R, DVD-R or a platter of a hard disk drive), on which is encoded computer-readable data 606. This computer-readable data 606 in turn comprises a set of computer instructions 604 configured to operate according to one or more of the principles set forth herein. In one such embodiment 602, the processor-executable instructions 604 may be configured to perform a method, such as at least some of the exemplary method 600 of FIG. 6 or the exemplary method 300 of FIG. 3, for example. In another such embodiment, the processor-executable instructions 604 may be configured to implement a system, such as at least some of the exemplary system 700 of FIG. 7, as described below. Many such computer-readable media may be devised by those of ordinary skill in the art that are configured to operate in accordance with the techniques presented herein.

FIG. 7 is a block diagram of an example of a computing system includes a managed rooms backbone with operational maintenance. The example system 700 includes a computing device 702. The computing device 702 includes a processing unit 704, a system memory 706, and a system bus 708. In some examples, the computing device 702 can be a gaming console, a personal computer (PC), an accessory console, a gaming controller, among other computing devices. Furthermore, the computing device 702 is not limited to a particular architecture. In examples, the computing architecture may be, for example, an x86 architecture, an Advanced RISC Machine (ARM) architecture, or any other computing architecture.

The system bus 708 couples system components including, but not limited to, the system memory 706 to the processing unit 704. The processing unit 704 can be any of various available processors. Dual microprocessors and other multiprocessor architectures also can be employed as the processing unit 704.

The system bus 708 can be any of several types of bus structure, including the memory bus or memory controller, a peripheral bus or external bus, and a local bus using any variety of available bus architectures known to those of ordinary skill in the art. The system memory 706 includes computer-readable storage media that includes volatile memory 710 and nonvolatile memory 712.

The BIOS, containing the basic routines to transfer information between elements within the computer 702, such as during start-up, is stored in nonvolatile memory 712. By way of illustration, and not limitation, nonvolatile memory 712 can include read-only memory (ROM), programmable ROM (PROM), electrically programmable ROM (EPROM), electrically erasable programmable ROM (EEPROM), or flash memory.

Volatile memory 710 includes random access memory (RAM), which acts as external cache memory. By way of illustration and not limitation, RAM is available in many forms such as static RAM (SRAM), dynamic RAM (DRAM), synchronous DRAM (SDRAM), double data rate SDRAM (DDR SDRAM), enhanced SDRAM (ESDRAM), SynchLink™ DRAM (SLDRAM), Rambus® direct RAM (RDRAM), direct Rambus® dynamic RAM (DRDRAM), and Rambus® dynamic RAM (RDRAM).

The computer 702 also includes other computer-readable media, such as removable/non-removable, volatile/non-volatile computer storage media. FIG. 7 shows, for example a disk storage 714. Disk storage 714 includes, but is not limited to, devices like a magnetic disk drive, floppy disk drive, tape drive, Jaz drive, Zip drive, flash memory card, or memory stick.

In addition, disk storage 714 can include storage media separately or in combination with other storage media including, but not limited to, an optical disk drive such as a compact disk ROM device (CD-ROM), CD recordable drive (CD-R Drive), CD rewritable drive (CD-RW Drive) or a digital versatile disk ROM drive (DVD-ROM). To facilitate connection of the disk storage devices 714 to the system bus 708, a removable or non-removable interface is typically used such as interface 716.

It is to be appreciated that FIG. 7 describes software that acts as an intermediary between users and the basic computer resources described in the suitable operating environment 700. Such software includes an operating system 718. Operating system 718, which can be stored on disk storage 714, acts to control and allocate resources of the computer 702. The operating system 718 as described herein is not limited to a particular operating system. In examples, the operating system may be, for example, a Windows-based operating system, an Android-based operating system, an iOS-based operating system, or any other operating system.

System applications 720 take advantage of the management of resources by operating system 718 through program modules 722 and program data 724 stored either in system memory 706 or on disk storage 714. It is to be appreciated that the disclosed subject matter can be implemented with various operating systems or combinations of operating systems.

The computer 702 may receive user commands or information through input devices 726. Input devices 726 include, but are not limited to, a pointing device, such as, a mouse, trackball, stylus, and the like, a keyboard, a microphone, a joystick, a satellite dish, a scanner, a TV tuner card, a digital camera, a digital video camera, a web camera, any suitable dial accessory (physical or virtual), and the like. In some examples, an input device can include Natural User Interface (NUI) devices. NUI refers to any interface technology that enables a user to interact with a device in a “natural” manner, free from artificial constraints imposed by input devices such as mice, keyboards, remote controls, and the like. In some examples, NUI devices include devices relying on speech recognition, touch and stylus recognition, gesture recognition both on screen and adjacent to the screen, air gestures, head and eye tracking, voice and speech, vision, touch, gestures, and machine intelligence. For example, NUI devices can include touch sensitive displays, voice and speech recognition, intention and goal understanding, and motion gesture detection using depth cameras such as stereoscopic camera systems, infrared camera systems, RGB camera systems and combinations of these. NUI devices can also include motion gesture detection using accelerometers or gyroscopes, facial recognition, three-dimensional (3D) displays, head, eye, and gaze tracking, immersive augmented reality and virtual reality systems, all of which provide a more natural interface. NUI devices can also include technologies for sensing brain activity using electric field sensing electrodes. For example, a NUI device may use Electroencephalography (EEG) and related methods to detect electrical activity of the brain. The input devices 726 connect to the processing unit 704 through the system bus 708 via interface ports 728. Interface ports 728 include, for example, a serial port, a parallel port, a game port, and a universal serial bus (USB).

Output devices 730 use some of the same type of ports as input devices 726. Thus, for example, a USB port may be used to provide input to the computer 702 and to output information from computer 702 to an output device 730.

Output adapter 732 is provided to illustrate that there are some output devices 730 like monitors, speakers, and printers, among other output devices 730, which are accessible via adapters. The output adapters 732 include, by way of illustration and not limitation, video and sound cards that provide a means of connection between the output device 730 and the system bus 708. It can be noted that other devices and systems of devices provide both input and output capabilities such as remote computing devices 734. The output devices 730 can also include any suitable fabrication device, such as a three-dimensional printer, which can generate three dimensional objects based on fabrication instructions transmitted from the computer 702. Output devices may also include an automated loom to create a fabric that passes colonnaded light that is controlled by the computer 702.

The computer 702 can be a server hosting various software applications in a networked environment using logical connections to one or more remote computers, such as remote computing devices 734. The remote computing devices 734 may be client systems configured with web browsers, PC applications, mobile phone applications, and the like. The remote computing devices 734 can be a personal computer, a server, a router, a network PC, a workstation, a microprocessor-based appliance, a mobile phone, a peer device or other common network node and the like, and typically includes many or all of the elements described relative to the computer 702. In some embodiments, the remote computing devices 734 can also include any suitable fabrication device that can manufacture a fabric such as a fabric that passes colonnaded light as described herein.

Remote computing devices 734 can be logically connected to the computer 702 through a network interface 736 and then connected via a communication connection 738, which may be wireless. Network interface 736 encompasses wireless communication networks such as local-area networks (LAN) and wide-area networks (WAN). LAN technologies include Fiber Distributed Data Interface (FDDI), Copper Distributed Data Interface (CDDI), Ethernet, Token Ring and the like. WAN technologies include, but are not limited to, point-to-point links, circuit switching networks like Integrated Services Digital Networks (ISDN) and variations thereon, packet switching networks, and Digital Subscriber Lines (DSL).

Communication connection 738 refers to the hardware/software employed to connect the network interface 736 to the bus 708. While communication connection 738 is shown for illustrative clarity inside computer 702, it can also be external to the computer 702. The hardware/software for connection to the network interface 736 may include, for exemplary purposes, internal and external technologies such as, mobile phone switches, modems including regular telephone grade modems, cable modems and DSL modems, ISDN adapters, and Ethernet cards.

The computer 702 can further include a radio 740. For example, the radio 740 can be a wireless local area network radio that may operate one or more wireless bands. For example, the radio 740 can operate on the industrial, scientific, and medical (ISM) radio band at 2.4 GHz or 5 GHz. In some examples, the radio 740 can operate on any suitable radio band at any radio frequency.

The computer 702 includes one or more modules 722, such as a managed rooms backbone 742. In some embodiments, the managed rooms backbone 742 enables an interconnect between managed devices and networks in a conference room of an organization. The organization may have any number of conference rooms, and each conference room can include a managed rooms backbone. In embodiments, the managed rooms backbone 742 enables seamless collaboration via the managed device functionality across one or more networks. The managed rooms backbone monitors communication between the managed devices and networks. The managed rooms backbone may also monitor communication between other conference rooms. In embodiments, the managed rooms backbone may function as an interface between a user and each managed device.

It is to be understood that the block diagram of FIG. 7 is not intended to indicate that the computing system 702 is to include all of the components shown in FIG. 7. Rather, the computing system 702 can include fewer or additional components not illustrated in FIG. 7 (e.g., additional applications, additional modules, additional memory devices, additional network interfaces, etc.).

It should also be noted that the different embodiments described herein can be combined in different ways. That is, parts of one or more embodiments can be combined with parts of one or more other embodiments. All of this is contemplated herein.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed is:

1. A device-implemented method for providing managed services, comprising:
determining each managed device of a plurality of managed devices available in a shared space;
determining health data for the shared space, wherein the health data describes properties of each managed device of the plurality of managed devices, the health data comprising update information, performance information, configuration information, and security information;
identifying a problem with at least one of the plurality of managed devices, the problem being identifiable based on analysis of the health data for the shared space; and
coordinating operational maintenance for the at least one of the plurality of managed devices to alleviate the problem, wherein coordinating operational maintenance comprises performing at least two of a software update, a configuration change, and uninstalling software.

2. The device-implemented method of claim 1, wherein coordinating operational maintenance comprises applying updates to the plurality of managed devices via a staged roll-out.

3. The device-implemented method of claim 2, wherein the staged rollout comprises assigning subsets of the plurality of managed devices to different rings and performing the operational maintenance on one ring at a time.

4. The device-implemented method of claim 3, wherein the rings comprise a staging ring, a general ring, and an executive ring.

5. The device-implemented method of claim 1, wherein coordinating operational maintenance comprises determining a risk assessment of the operational maintenance.

6. The device-implemented method of claim 1, wherein coordinating operational maintenance comprises identifying a first common task to be performed on more than one of the plurality of managed devices.

7. The device-implemented method of claim 6, wherein the first common task is performed for each one of the plurality of managed devices.

8. The device-implemented method of claim 6, wherein coordinating operational maintenance comprises a identifying a second common task to be performed on the more than one of the plurality of managed devices, and wherein the first common task and the second common task are combined into a blended task.

9. The device-implemented method of claim 1, wherein determining the health data for the shared space comprises analyzing hardware signals from each of the plurality of managed devices.

10. The device-implemented method of claim 1, wherein the update information for each managed device identifies a particular update version executing on the managed device.

11. The device-implemented method of claim 1, wherein the operational maintenance comprises maintaining an operational configuration for each managed device of the plurality of managed devices.

12. The device-implemented method of claim 1, wherein operational maintenance comprises maintaining security settings for each managed device of the plurality of managed devices.

13. A management services system to provide operational maintenance, the system comprising:
a network interface to receive health data from a monitored system comprising a plurality of managed devices available in a shared space; and
one or more processing devices configured to:
determine each managed device of the plurality of managed devices;
determine a health data for the shared space, wherein the health data describes properties of each managed device of the plurality of managed devices, the health data comprising update information, performance information, configuration information, and security information;
identify a problem with at least one of the plurality of managed devices, the problem being identifiable based on analysis of the health data for the shared space; and
coordinating operational maintenance for the at least one of the plurality of managed devices to alleviate the problem, wherein coordinating operational maintenance comprises performing at least two of a software update, a configuration change, and uninstalling software.

14. The system of claim 13, wherein coordinating operational maintenance comprises applying updates to the plurality of managed devices via a staged roll-out, wherein the staged rollout comprises assigning subsets of the plurality of managed devices to different rings and performing the operational maintenance on one ring at a time.

15. The system of claim 13, wherein coordinating operational maintenance comprises identifying a first common task to be performed on more than one of the plurality of managed devices.

16. The system of claim 15, wherein coordinating operational maintenance comprises a identifying a second common task to be performed on the more than one of the plurality of managed devices, and wherein the first common task and the second common task are combined into a blended task.

17. The system of claim 13, wherein identifying the problem with at least one of the plurality of managed devices comprises inputting the health data to a machine learning engine.

18. The system of claim 15, wherein coordinating operational maintenance comprises performing one or more tasks on the monitored system, wherein the one or more tasks are remediation tasks output by the machine learning engine.

19. A tangible, computer-readable storage medium comprising instructions that, in response to an execution by a processor, cause the processor to:
determine each managed device of a plurality of managed devices available in a shared space;
determine health data for the shared space, wherein the health data describes properties of each managed device of the plurality of managed devices, the health data comprising update information, performance information, configuration information, and security information;
identify a problem with at least one of the plurality of managed devices, the problem being identifiable based on analysis of the health data for the shared space; and
coordinating operational maintenance for the at least one of the plurality of managed devices to alleviate the problem, wherein coordinating operational maintenance comprises performing at least two of a software update, a configuration change, and uninstalling software.

20. The tangible, computer-readable storage medium of claim 19, wherein coordinating operation maintenance comprises identifying a combination of remediation tasks to be performed on two or more of the managed devices.
